(19) United States
(12) Patent Application Publication
(10) Pub. No.: US 2024/0090776 A1
(43) Pub. Date: Mar. 21, 2024

(54) APPARATUS AND METHOD OF NONINVASIVELY AND SEPARATELY MEASURING LUNG VENTILATION AND CARDIAC BLOOD FLOW COMPONENTS

(71) Applicant: BILAB CO., LTD., Gyeonggi-do (KR)

(72) Inventors: Geuk Young Jang, Gyeonggi-do (KR); Hun Wi, Seoul (KR)

(21) Appl. No.: 18/263,587

(22) PCT Filed: Jun. 3, 2021
(86) PCT No.: PCT/KR2021/006962

§ 371 Date: Jul. 31, 2023

(30) Foreign Application Priority Data
Feb. 4, 2021 (KR) 10-2021-0016253

(51) Int. Cl. A61B 5/0205; A61B 5/00; A61B 5/0265; A61B 5/08

(52) U.S. Cl. CPC A61B 5/0205; A61B 5/0265; A61B 5/0809; A61B 5/7257; A61B 5/7278; A61B 5/7425; A61B 5/0536

(57) ABSTRACT

The present invention relates to an apparatus and a method of noninvasively separating and measuring a lung ventilation component and a cardiac blood flow component, and more specifically, to an apparatus and a method for extracting shape-reference voltage waveforms associated with lung ventilation and cardiac blood flow through principal component analysis (PCA) and independent component analysis (ICA), respectively, from time series voltage data acquired from a subject, decomposing the lung ventilation component and the cardiac blood flow component for each voltage channel using the extracted shape-reference voltage waveforms, thereby noninvasively, simultaneously, and continuously measuring tidal volume and stroke volume, respectively, from the decomposed lung ventilation component and cardiac blood flow component.

### FIELD OF THE INVENTION

The present invention relates to an apparatus and a method of noninvasively separating and measuring a lung ventilation component and a cardiac blood flow component, and more specifically, to an apparatus and a method for extracting shape-reference voltage waveforms associated with lung ventilation and cardiac blood flow through principal component analysis (PCA) and independent component analysis (ICA), respectively, from time series voltage data acquired from a subject, decomposing the lung ventilation component and the cardiac blood flow component for each voltage channel using the extracted shape-reference voltage waveforms, thereby noninvasively, simultaneously, and continuously measuring tidal volume and stroke volume, respectively, from the decomposed lung ventilation component and cardiac blood flow component.

### BACKGROUND ART

Stroke volume and cardiac output of a heart are major indicators for identifying the hemodynamic state of a patient, and tidal volume is a key indicator capable of detecting a low ventilation state of a patient with symptoms of respiratory degradation.

In general, a transpulmonary thermodilution (TPTD) method is representative in a method of measuring stroke volume. In the TPTD method, stroke volume can be measured by inserting a catheter having a built-in temperature sensor into an artery of a subject, injecting physiological saline solution into a central vein, and then measuring the amount of change in temperature according to the injection of the physiological saline solution through the catheter.

However, in the TPTD method, there is an inconvenience of requiring insertion of the catheter into a body, and there is a problem in that whenever stroke volume is measured, the injection of the physiological saline solution is needed, so that the stroke volume cannot be measured continuously.

In addition, tidal volume may be measured by an artificial respirator intubated into a patient, but there is a problem in that the tidal volume cannot be measured for a non intubated patient. In case of a non intubation patient, the tidal volume can be measured by using a mask, but it is difficult to monitor the tidal volume for a long time. That is, it is common that the stroke volume and the tidal volume should be individually measured through different invasive or constraining means.

If stroke volume and tidal volume can be continuously and simultaneously measured, it is possible to effectively monitor the cardiopulmonary function of a patient by integrating the stroke volume and the tidal volume.

Recently, active researches have been conducted on an EIT technology, in which a current is injected through a plurality of electrical impedance tomography (EIT) electrodes attached to the chest of a patient, voltage data corresponding to the injected current are measured, then an EIT image for an internal body of a patient is reconstructed by using the measured voltage data, and finally lung ventilation (airflow) or cardiac blood flow due to breathing is noninvasively monitored based on the reconstructed image.

Generally EIT technology is used to acquire voltage data from a patient while performing machine ventilation to the patient and to restore a local ventilation (lung ventilation) image inside the lung.

However, the acquired voltage data is affected by cardiac blood flow (blood flow), and since the affection of cardiac blood flow in the measured voltage data is much weaker than that of a ventilation, it has been difficult to restore an image of cardiac blood flow in the conventional EIT technology.

Meanwhile, in the conventional EIT technology, the measured voltage data was first reconstructed into image data, and the lung ventilation component and the cardiac blood flow component were separated or decomposed from the image data, and thus since in this case, loss or distortion of data is occurred in the process of reconstructing the image data, it is difficult to accurately separate or decompose a cardiac blood flow component from the reconstructed image data.

If the voltage data measured from the patient can be divided or decomposed at first into the voltage data corresponding to lung ventilation and the voltage data corresponding to cardiac blood flow, the reconstructed images of lung ventilation and cardiac blood flow may be more precisely reconstructed, and the tidal volume and the stroke volume may be more precisely and continuously measured at the same time.

Therefore, the present invention presents a method of extracting a shape-reference voltage waveforms related to lung ventilation and cardiac blood flow respectively from time series voltage data acquired from a subject through PCA (Principal Component Analysis) and ICA (Independent Component Analysis), separating/decomposing a lung ventilation component and a cardiac blood flow component by using the extracted shape-reference voltage waveforms, and then reconstructing each component into an image, or noninvasively and continuously measuring tidal volume and stroke volume.

Hereinafter, prior arts existing in the technical field of the present invention are briefly described, and distinctive technical aspects that are achieved by the present invention in comparison with the prior arts are described with reference to the prior arts.

First, U.S. Pat. No. 8,321,007 B1 (Feb. 9, 2010) relates to an apparatus and a method for determining the characteristics of a functional lung for each region of interest, by reconstructing an EIT image of a lung by using voltage data measured from a patient who performs a machine ventilation, and calculating a ratio of impedance change to a plurality of regions of interest set on the reconstructed EIT image, to the entire impedance change of the restored EIT image.

The above prior art describes only the technical feature of reconstructing a general EIT image, however the present invention is to respectively separate/decompose a time series voltage data continuously obtained from a subject into voltage data for lung ventilation and cardiac blood flow through a PCA and ICA, and reconstruct lung ventilation and cardiac blood flow images, or noninvasively and continuously and simultaneously measure the tidal volume and stroke volume. The above prior art does not describe, suggest, or imply any of these technical features of the present invention.

U.S. Pat. No. 8,764,667 B1 (Jul. 1, 2014) relates to a sleep monitoring method and system, and more particularly, to the sleep monitoring method and system for calculating cardiac output by injecting a high frequency signal into any one of two electrodes attached to a patient during sleep and determining a phase shift of a high frequency signal inputted from the other electrodes corresponding to the injected high frequency signal.

The above prior art is to calculate a cardiac output by using a high-frequency signal acquired from a patient during sleep, is not to separate/decompose time-series voltage data into voltage data for lung ventilation and cardiac blood flow through principal component analysis and independent component analysis, as provided in the present invention, and is not to reconstruct lung ventilation and cardiac blood flow images by using the separated/decomposed voltage data or not to measure non-invasively, simultaneously, and continuously tidal volume and stroke volume. So, the prior art does not describe the above features, and thus both inventions have remarkable differences.

### DETAILED DESCRIPTION OF THE INVENTION

### Technical Problem

The present invention is created to solve the aforementioned problems, and it is an objective of the present invention to provide an apparatus and a method of measuring non-invasively and continuously both tidal volume and stroke volume by pre-separating lung ventilation component by respiration and a cardiac blood flow component due to heartbeat, before reconstructing time series voltage data into images, in which the time series voltage data are acquired from a subject through a plurality of voltage channels.

Furthermore, it is an objective of the present invention to provide an apparatus and method of extracting a shape-reference voltage waveform corresponding to lung ventilation and a shape-reference voltage waveform corresponding to cardiac blood flow from the acquired time series voltage data by using principal component analysis and independent component analysis, computing scale factors and offsets related to lung ventilation and cardiac blood flow for the voltage data for each voltage channel by using the extracted shape-reference voltage waveforms, and separating/decomposing the time series voltage data into the voltage data for lung ventilation and cardiac blood flow by using the calculated scale factors and offsets.

In addition, it is an objective of the present invention to provide an apparatus and a method of simultaneously measuring the tidal volume and the stroke volume, by extracting a respiratory volume signal (RVS) and a cardiac volume signal (CVS) from each separated time series voltage data for lung ventilation and cardiac blood flow, and calculating a difference between valley-to-peak values of the respiratory volume signal and the cardiac volume signal.

In addition, it is an objective of the present invention to provide an apparatus and a method of measuring stroke volume variation (SVV) by using a maximum stroke volume and a minimum stroke volume among stroke volumes measured in a plurality of breathing cycles by using the respiratory volume signal and the cardiac volume signal.

Furthermore, it is an objective of the present invention to provide an apparatus and a method for monitoring a cardiopulmonary function of a subject by providing the measured tidal volume, stroke volume, stroke volume variation, or a combination thereof.

### Problem Solving Means

It is characterized in that an apparatus of noninvasively and separately measuring a lung ventilation component and a cardiac blood flow component, according to one embodiment of the present invention, is configured to comprise: a voltage data acquisition unit configured to acquire a time series voltage data from a subject through a plurality of voltage channels, a voltage data decomposition unit configured to decompose the acquired time series voltage data into voltage data of lung ventilation and cardiac blood flow, which are respectively the lung ventilation component and the cardiac blood flow component, and a measurement unit configured to measure variations in lung ventilation and in cardiac blood flow from the decomposed voltage data, wherein the time series voltage data are composed of a linear weighted sum of impedance variations caused by a plurality of physiological activities comprising lung ventilation and cardiac blood flow.

It is characterized in that wherein the voltage data decomposition unit is further configured to comprise a shape-reference voltage waveform extraction unit configured to extract shape-reference voltage waveforms associated with lung ventilation and cardiac blood flow through principal component analysis (PCA) and independent component analysis (ICA) from the time series voltage data acquired from the subject, wherein the lung ventilation component and the cardiac blood flow component are decomposed for each voltage channel by using the extracted shape-reference voltage waveforms, and thereby the acquired time series voltage data is decomposed into the voltage data for lung ventilation and cardiac blood flow, respectively.

It is characterized in that wherein the apparatus is further configured to comprise an image reconstruction unit configured to reconstruct the decomposed voltage data for lung ventilation and cardiac blood flow into images of lung ventilation and cardiac blood flow, respectively, wherein the variations in lung ventilation and cardiac blood flow are noninvasively, simultaneously, and continuously measured from the reconstructed images of lung ventilation and cardiac blood flow.

It is characterized in that wherein the measurement unit is further configured to extract a respiratory volume signal and a cardiac volume signal from the reconstructed images of lung ventilation and cardiac blood flow, or the decomposed voltage data of lung ventilation and cardiac blood flow, respectively, and noninvasively, simultaneously, and continuously measure tidal volume and stroke volume, thereby measuring the variations in lung ventilation and cardiac blood flow.

It is characterized in that wherein the shape-reference voltage waveform extraction unit is further configured to comprise a lung ventilation shape-reference voltage waveform extraction unit configured to choose a principal component in descending order of singular values as results of applying principal component analysis on the acquired time series voltage data, and extract the chosen principal component as a shape-reference voltage waveform associated with lung ventilation, and a cardiac blood flow shape-reference voltage waveform extraction unit configured to extract a plurality of independent components by applying independent component analysis on a plurality of principal components excluding the chosen principal component, and extract independent components associated with heartbeats among the plurality of the extracted independent components as a shape-reference voltage waveforms associated with cardiac blood flow.

It is characterized in that wherein the voltage data decomposition unit is further configured to comprise a weight computing unit configured to compute weights comprising scale factors and offsets for lung ventilation and cardiac blood flow for each voltage channel associated with the acquired time series voltage data by using the extracted shape-reference voltage waveforms of lung ventilation and cardiac blood flow, wherein the voltage data of lung ventilation and cardiac blood flow from the voltage data are computed for each voltage channel by using the weights associated with lung ventilation and cardiac blood flow computed for each voltage channel, so that the acquired time series voltage data are decomposed into the voltage data of lung ventilation and cardiac blood flow.

It is characterized in that wherein the cardiac blood flow shape-reference waveform extraction unit is further configured to apply a Fast Fourier Transform (FFT) on each of the plurality of the extracted independent components, and obtain a frequency spectrum for each extracted independent component, and choose the independent component for the frequency spectrum having the biggest energy within the fundamental frequency range of the heartbeat rate, so that the cardiac blood flow shape-reference voltage waveform is extracted.

It is characterized in that wherein the weight computing unit is further configured to represent the voltage data for each voltage channel of the acquired time series voltage data as a weighted sum of the extracted lung ventilation shape-reference voltage waveform and the cardiac blood flow shape-reference voltage waveform, and compute weights of lung ventilation and cardiac blood flow by applying a least square method for each voltage channel from the represented weighted sum.

It is characterized in that wherein the respiratory volume signal is extracted by summing all the voltage data for region of interests preset in a lung region in the decomposed voltage data of lung ventilation or pixel values for region of interests preset in a lung region in the reconstructed image of lung ventilation, respectively, and wherein the cardiac volume signal is extracted by summing all the voltage data for region of interests preset in a heart region in the decomposed voltage data of cardiac blood flow or pixel values for region of interests preset in the heart region in the reconstructed image of cardiac blood flow, respectively.

It is characterized in that wherein the tidal volume is measured for each breathing cycle by computing a valley-to-peak value of each breathing cycle detected from the extracted respiratory volume signal, wherein the stroke volume is measured for each heartbeat cycle by computing a valley-to-peak value of each heartbeat cycle detected from the extracted cardiac volume signal, and wherein the breathing cycle and the heartbeat cycle are extracted by detecting continuous occurrence of valley-peak-valley in the extracted each respiratory volume signal and cardiac volume signal.

It is characterized in that wherein the measurement unit is further configured to comprise mutually overlapping the extracted respiratory volume signal and cardiac volume signal over time and measuring a change in the stroke volumes according to a plurality of preset breathing cycles, by using a maximum value and a minimum value of the measured stroke volumes in the plurality of preset breathing cycles.

It is characterized in that a method of noninvasively and separately measuring a lung ventilation component and a cardiac blood flow component, according to one embodiment of the present invention, comprises acquiring time series voltage data from a subject through a plurality of voltage channels, decomposing the acquired time series voltage data into voltage data of lung ventilation and cardiac blood flow, which are respectively the lung ventilation component and the cardiac blood flow component, and measuring variations in lung ventilation and in cardiac blood flow from the decomposed voltage data, wherein the time series voltage data are composed of a linear weighted sum of impedance variations caused by a plurality of physiological activities comprising lung ventilation and cardiac blood flow.

It is characterized in that wherein the decomposing of the voltage data further comprises extracting shape-reference voltage waveforms associated with lung ventilation and cardiac blood flow through principal component analysis (PCA) and independent component analysis (ICA) from the time series voltage data acquired from the subject, wherein the lung ventilation component and the cardiac blood flow component are decomposed for each voltage channel by using the extracted shape-reference voltage waveforms, and thereby the acquired time series voltage data is decomposed into the voltage data for lung ventilation and cardiac blood flow, respectively.

It is characterized in that wherein the method further comprises reconstructing the decomposed voltage data for lung ventilation and cardiac blood flow into images of lung ventilation and cardiac blood flow, respectively, wherein the variations in lung ventilation and cardiac blood flow are noninvasively, simultaneously, and continuously measured from the reconstructed images of lung ventilation and cardiac blood flow.

It is characterized in that wherein the measuring of variations further comprises extracting a respiratory volume signal and a cardiac volume signal from the reconstructed images of lung ventilation and cardiac blood flow, or the decomposed voltage data of lung ventilation and cardiac blood flow, respectively, and noninvasively, simultaneously, and continuously measuring tidal volume and stroke volume, thereby measuring the variations in lung ventilation and cardiac blood flow.

It is characterized in that wherein the extracting of the shape-reference voltage waveforms further comprises choosing/selecting a principal component in descending order of singular values as results of applying principal component analysis on the acquired time series voltage data, and extracting the chosen principal component as a shape-reference voltage waveform associated with lung ventilation, and extracting a plurality of independent components by applying independent component analysis on a plurality of principal components excluding the chosen principal component, and extracting independent components associated with heartbeats among the plurality of the extracted independent components as a shape-reference voltage waveforms associated with cardiac blood flow.

It is characterized in that wherein the decomposing of the voltage data further comprises computing weights comprising scale factors and offsets for lung ventilation and cardiac blood flow for each voltage channel associated with the acquired time series voltage data by using the extracted shape-reference voltage waveforms of lung ventilation and cardiac blood flow, wherein the voltage data of lung ventilation and cardiac blood flow from the voltage data are computed for each voltage channel by using the weights associated with lung ventilation and cardiac blood flow computed for each voltage channel, so that the acquired time series voltage data are decomposed into the voltage data of lung ventilation and cardiac blood flow.

It is characterized in that wherein the extracting of the cardiac blood flow shape-reference waveform further comprises applying a Fast Fourier Transform (FFT) on each of the plurality of the extracted independent components, and obtaining a frequency spectrum for each extracted independent component, and choose the independent component for the frequency spectrum having the biggest energy within the fundamental frequency range of the heartbeat rate, so that the cardiac blood flow shape-reference voltage waveform is extracted.

It is characterized in that wherein the computing of weights further comprises representing the voltage data for each voltage channel of the acquired time series voltage data as a weighted sum of the extracted lung ventilation shape-reference voltage waveform and the cardiac blood flow shape-reference voltage waveform, and computing weights of lung ventilation and cardiac blood flow by applying a least square method for each voltage channel from the represented weighted sum.

It is characterized in that wherein the measuring of the variations further comprises mutually overlapping the extracted respiratory volume signal and cardiac volume signal over time and measuring a change in the stroke volumes according to a plurality of preset breathing cycles, by using a maximum value and a minimum value of the measured stroke volumes in the plurality of preset breathing cycles.

### Effects of the Invention

As described above, the apparatus and method of noninvasively separating and measuring a lung ventilation component and a cardiac blood flow component according to the present invention have effects of being capable of precisely, noninvasively and simultaneously, and continuously measuring tidal volume and stroke volume according to a respiration and a cardiac output of a subject, respectively, by extracting shape-reference voltage waveforms associated with lung ventilation and cardiac blood flow, respectively, through principal component analysis (PCA) and independent component analysis (ICA) from time series voltage data acquired from a subject, and separating the lung ventilation component and the cardiac blood flow component for each voltage channel by using the extracted shape-reference voltage waveforms.

Furthermore, the apparatus and method according to the present invention have additional effects of being capable of effectively monitoring the cardiopulmonary function of a subject by reconstructing the measured tidal volume and stroke volume into images and outputting the reconstructed images on a display or by outputting the measured tidal volume and stroke volume to graph/text (numerical values) on the display.

### BRIEF DESCRIPTION OF THE DRAWINGS

FIG. 1 is a conceptual diagram illustrating an apparatus and a method of noninvasively and separately measuring lung ventilation component and a cardiac blood flow component according to an embodiment of the present invention.

FIG. 2 is a view illustrating a time series voltage data obtained from a subject according to an embodiment of the present invention.

FIG. 3 is a diagram illustrating a process of separating a time series voltage data into lung ventilation component and a cardiac blood flow component according to an embodiment of the present invention.

FIG. 4 is a diagram illustrating a distribution of singular values obtained by performing principal component analysis according to an embodiment of the present invention.

FIG. 5 is a diagram illustrating a method of extracting a cardiac blood flow shape-reference waveform by performing independent component analysis according to an embodiment of the present invention.

FIG. 6 is a view illustrating lung ventilation component according to an embodiment of the present invention.

FIG. 7 is a view illustrating cardiac blood flow components according to an embodiment of the present invention.

FIG. 8 is a diagram illustrating voltage data, lung ventilation, and cardiac blood flow components for each voltage channel according to an embodiment of the present invention.

FIG. 9 is a diagram illustrating reconstructed image of lung ventilation and a respiratory volume signal according to an embodiment of the present invention.

FIG. 10 is a diagram illustrating a cardiac blood flow image and a cardiac volume signal according to an embodiment of the present invention.

FIG. 11 is a diagram illustrating overlapping a respiratory volume signal and a cardiac volume signal of a normal subject and an abnormal subject according to an embodiment of the present disclosure.

FIG. 12 is a block diagram illustrating a configuration of an apparatus of noninvasively and separately measuring lung ventilation component and a cardiac blood flow component according to an embodiment of the present invention.

FIG. 13 is a block diagram illustrating a configuration of a voltage data decomposition unit according to an embodiment of the present invention.

FIG. 14 is a flowchart illustrating a procedure of noninvasively and separately measuring lung ventilation component and a cardiac blood flow component according to an embodiment of the present invention.

FIG. 15 is a flowchart illustrating a detailed procedure of separating voltage data according to an embodiment of the present invention.

FIG. 16 is a view showing an accuracy of tidal volume according to an embodiment of the present invention.

FIG. 17 is a view showing an accuracy of stroke volume according to an embodiment of the present invention.

### BEST FORM FOR IMPLEMENTATION OF THE INVENTION

Hereinafter, preferred embodiments of the apparatus and method of noninvasively and separately measuring a lung ventilation component and a cardiac blood flow component of the present invention are described in detail with reference to the accompanying drawings. The identical reference numerals in the drawings indicate the same components. Furthermore, specific structural or functional descriptions regarding to the preferred embodiments of the present invention are only provided for the purpose of illustratively explaining the embodiments according to the present invention. Unless otherwise defined, all terms used herein, including technical or scientific terminologies, have the same meanings as commonly understood by those ordinary skilled in the technical field to which the present disclosure pertains. Terms such as those defined in commonly used dictionaries, should be interpreted to have the meanings consistent with their meanings on the context of the relevant art, and it is desirable not to interpret the terms in an idealized or overly formal sense unless explicitly defined in this specification.

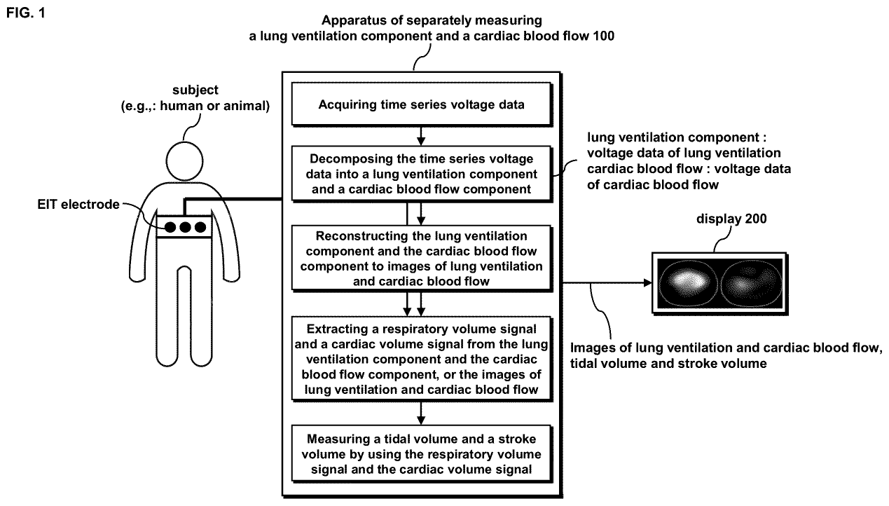

FIG. 1 is a conceptual diagram illustrating an apparatus and a method of noninvasively and separately measuring lung ventilation component and a cardiac blood flow component according to an embodiment of the present invention.

As shown in FIG. 1, an apparatus 100 of noninvasively and separately measuring a lung ventilation component and a cardiac blood flow component according to an embodiment of the present invention (hereinafter, referred to as an apparatus of separately measuring a lung ventilation component and a cardiac blood flow component) is configured to separate/decompose time series voltage data acquired from a subject into a lung ventilation component (i.e., voltage data for lung ventilation) and a cardiac blood flow component (i.e., voltage data for cardiac blood flow), reconstruct the separated/decomposed voltage data by the lung ventilation and the cardiac blood flow into an image for the lung ventilation (reconstructed image of lung ventilation) and an image for the cardiac blood flow (cardiac blood flow image), and then perform a function by noninvasively, continuously and simultaneously measuring and providing tidal volume and stroke volume by using the separated voltage data for lung ventilation and cardiac blood flow or the reconstructed images of lung ventilation and cardiac blood flow.

Wherein the lung ventilation refers to an airflow (i.e., ventilation) caused by respiration of a subject, and the cardiac blood flow refers to a blood flow caused by heartbeat of the subject.

The time series voltage data is acquired by continuously measuring voltages through a plurality of electrodes attached to the chest of a subject. In this case, the voltages are measured through the plurality of electrodes by sequentially selecting an adjacent pair of electrodes from among the plurality of electrodes and sequentially injecting current to the selected electrode pair. That is, the time series voltage data are acquired by measuring voltages (i.e., impedance variations) corresponding to the injected current.

For example, when the plurality of electrodes are 16, and the currents between all 16 adjacent electrode pairs are sequentially injected, 256 (=16×16) voltage measurement values are acquired, and these measurement voltages constitutes one time scan for the chest of the subject.

In this case, three measured voltage data acquired from three adjacent electrode pairs including the current injection electrode among the measured voltage data are discarded since they are affected by the contact impedance of the current injection electrodes. For example, the time series voltage data including 208(16×(16−3)) consecutive measurement voltage data from consecutive scans at 100 frames/s (i.e., 100 scans per second) are finally acquired.

Wherein, the time series voltage data are acquired through each of electrodes of measuring voltages according to the current injection, and each of the electrodes becomes a voltage channel for acquiring the voltage data. That is, the time series voltage data are acquired (or obtained) through voltage channels formed through a plurality of electrodes attached to the chest of the subject. Note that each of these time series is called a voltage channel.

The length of time series for each voltage channel is N=100×T (time), in the above example.

The apparatus 100 of separately measuring a lung ventilation component and a cardiac blood flow component is configured to first separate/decompose the acquired time series voltage data into the voltage data for lung ventilation and cardiac blood flow, respectively, in order to noninvasively, simultaneously and continuously measure tidal volume and stroke volume.

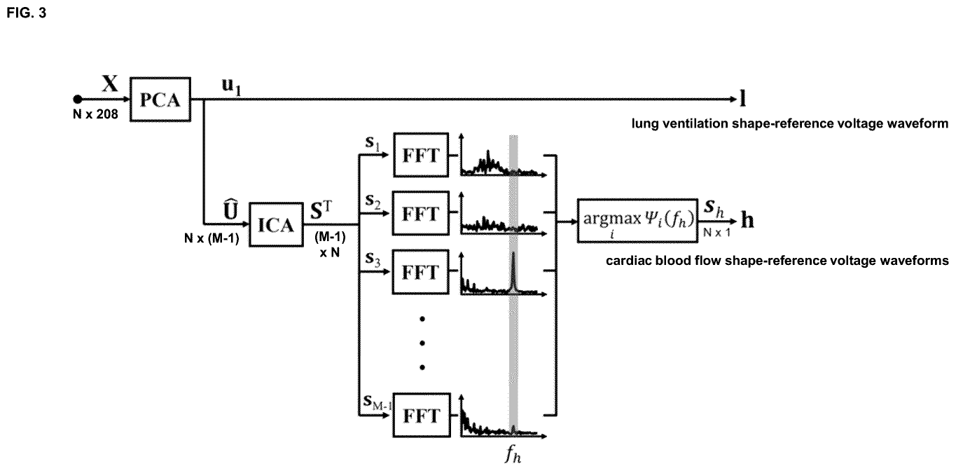

The separating/decomposing of the voltage data for the lung ventilation and the cardiac blood flow is performed by extracting a shape-reference waveform associated with lung ventilation(hereinafter, referred to as a lung ventilation shape-reference waveform) and a shape-reference waveform associated with cardiac blood flow (hereinafter, referred to as a cardiac blood flow shape-reference waveform) from the acquired time series voltage data through principal component analysis and independent component analysis, and utilizing the extracted lung ventilation shape-reference waveform and cardiac blood flow shape-reference waveform. Furthermore, the separating/decomposing of the acquired time series voltage data into the voltage data for lung ventilation and cardiac blood flow is described in detail with reference to FIG. 3.

In addition, the apparatus 100 of separately measuring a lung ventilation component and a cardiac blood flow component is configured to reconstruct the separated/decomposed voltage data for lung ventilation and cardiac blood flow into the images of lung ventilation and cardiac blood flow, respectively, through a linearized image reconstruction algorithm using a sensitivity matrix derived from a lead field theory.

In this case, the apparatus 100 of separately measuring a lung ventilation component and a cardiac blood flow component is configured to consecutively reconstruct the images of lung ventilation and cardiac blood flow, by separating/decomposing the consecutively acquired time series voltage data into the voltage data for lung ventilation and cardiac blood flow, respectively, and reconstructing the separated voltage data for lung ventilation and cardiac blood flow into the images for lung ventilation and cardiac blood flow.

In addition, the apparatus 100 of separately measuring a lung ventilation component and a cardiac blood flow component is configured to extract a respiratory volume signal (RVS) and a cardiac volume signal (CVS) from the reconstructed images of lung ventilation and cardiac blood flow, or the separated/decomposed voltage data for lung ventilation and cardiac blood flow, respectively.

In this case, the respiratory volume signal is extracted as sum of all the voltage data within preset lung ROIs (region of interests) in the separated/decomposed voltage data of lung ventilation or sum of all the pixel values within the preset lung ROIs in the reconstructed image of lung ventilation.

In addition, the cardiac volume signal is extracted as sum of all the voltage data within the preset heart ROIs for the separated/decomposed voltage data of cardiac blood flow or sum of all the pixel values within the preset heart ROIs in the reconstructed image of cardiac blood flow.

The apparatus 100 of separately measuring a lung ventilation component and a cardiac blood flow component is configured to noninvasively, simultaneously, and continuously measure variations (changes) in lung ventilation and cardiac blood flow from the extracted respiratory volume signal and cardiac volume signal. A change/variation in lung ventilation refers to tidal volume, and a change/variation in cardiac blood flow refers to stroke volume.

The apparatus 100 of separately measuring a lung ventilation component and a cardiac blood flow component is configured to further comprise measuring stroke volume variation from the extracted cardiac volume signal.

The tidal volume is computed with valley-to-peak values for each breathing cycle detected from the extracted respiratory volume signal. That is, the tidal volume is computed as an absolute value of a difference between the peak value and the valley value. The breathing cycle is detected by inspecting that the valley-peak-valley consecutively appears in the extracted respiratory volume signal.

The stroke volume is computed with valley-to-peak values for each heartbeat cycle detected from the extracted cardiac volume signal. The heartbeat cycle is detected by inspecting that the valley-peak-valley consecutively appears in the extracted cardiac volume signal.

The apparatus 100 of separately measuring a lung ventilation component and a cardiac blood flow component is configured to output the reconstructed images of lung ventilation and cardiac blood flow and the measured tidal volume and stroke volume; or the measured tidal volume, stroke volume and the stroke volume variation on a display 200. The tidal volume, stroke volume and stroke volume variation are reconstructed into graphs or images that are changed according to a flow of time, and then the reconstructed graphs or images are outputted on the display 200.

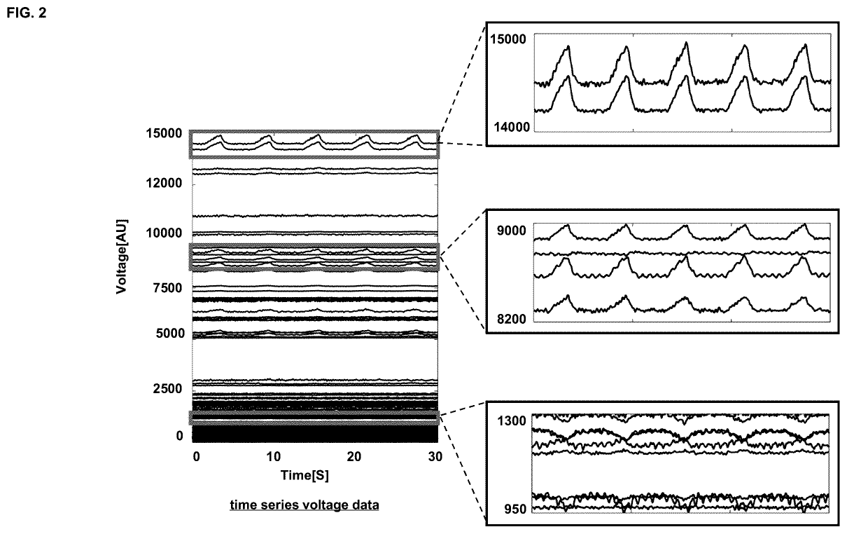

FIG. 2 is a view illustrating a time series voltage data obtained from a subject according to an embodiment of the present invention.

As shown in FIG. 2, the time series voltage data acquired from the subject according to an embodiment of the present invention is acquired by injecting current to sequentially selected adjacent electrode pairs among a plurality of electrodes attached to the chest of the subject during a specific time (e.g., 1 second), respectively and measuring voltages corresponding to the injected current through the remaining electrodes.

The time series voltage data are consecutively acquired during each specific time.

FIG. 3 is a diagram illustrating a process of separating a time series voltage data into lung ventilation component and a cardiac blood flow component according to an embodiment of the present invention.

As shown in FIG. 3, the apparatus 100 of separately measuring a lung ventilation component and a cardiac blood flow component according to an embodiment of the present invention is configured to extract the lung ventilation shape-reference voltage waveform and the cardiac blood flow shape-reference voltage waveform from the time series voltage data acquired from the subject by using the principal component analysis and the independent component analysis. In addition, the apparatus 100 of separately measuring a lung ventilation component and a cardiac blood flow component is configured to separate/decompose the acquired (obtained) time series voltage data into the lung ventilation voltage data (lung ventilation component) and the cardiac blood flow voltage data (a cardiac blood flow component) using the extracted lung ventilation shape-reference voltage waveform and cardiac blood flow shape-reference voltage waveform.

Hereinafter, the acquiring of time series voltage data using 16 electrodes is to be described as an example.

The time series voltage data for the obtained 208 voltage channels is represented by N×208 data matrix X, as shown in [Equation 1] below.

$$X = ( \begin{matrix}↑ & ↑ & … & ↑ \\ {x}_{1} & {x}_{2} & … & {x}_{208} \\ ↓ & ↓ & … & ↓\end{matrix} ) \qquad [ Equation 1 ]$$

Wherein, xᵢ for i=1, 2, . . . , 208 is the N×1 data vector of the ith voltage channel measured in the scan of N times.

In addition, the apparatus 100 of separately measuring a lung ventilation component and a cardiac blood flow component is configured to extract the first principal component by applying the principal component analysis to the matrix X according to the time axis (i.e., the scan order), thereby extracting the lung ventilation shape-reference waveform associated with lung ventilation.

The choice of the first principal component is based on the observation that lung ventilation produces the largest change in the time series voltage data (i.e., matrix X).

The principal component analysis is implemented using the singular value decomposition (SVD) of XX^{T} with respect to the matrix X represented by [Equation 2].

$${XX}^{T} = {\sum }_{j = 1}^{N} {λ}_{j} {u}_{j} {u}_{j}^{T} \qquad [ Equation 2 ]$$

where λⱼ for j=1, 2, . . . , N are the singular values in descending order for the matrix X, and uⱼ are the singular vectors corresponding to the singular values.

In addition, the apparatus 100 of separately measuring a lung ventilation component and a cardiac blood flow component is configured to extract the lung ventilation shape-reference waveform represented by [Equation 3] by choosing a single principal component in the order in which the singular value is greater through the principal component analysis.

l=u₁ [Equation 3]

Wherein, l is a shape-reference waveform of lung ventilation, and u₁ represents one principal component chosen in a descending order of the singular values among a plurality of principal components extracted through the principal component analysis.

In addition, the apparatus 100 of separately measuring a lung ventilation component and a cardiac blood flow component is configured to form Û of a matrix of N×(M−1) from which the selected (extracted) principal component (u₁) is removed among the plurality of extracted principal components, as shown in [Equation 4].

$$\hat{U} = ( \begin{matrix}↑ & ↑ & … & ↑ \\ {u}_{2} & {u}_{3} & … & {u}_{M} \\ ↓ & ↓ & … & ↓\end{matrix} ) \qquad [ Equation 4 ]$$

Wherein, the removing of the selected (extracted) principal component is to suppress the influence of lung ventilation, and M is less than 208.

In addition, when the independent component analysis is applied on the constructed matrix Û, the signal source matrix S for a plurality of independent components is represented by [Equation 5].

$$\begin{matrix}{S}^{T} = W {\hat{U}}^{T} \\ S = ( \begin{matrix}↑ & ↑ & … & ↑ \\ {s}_{1} & {s}_{2} & … & {s}_{M - 1} \\ ↓ & ↓ & … & ↓\end{matrix} )\end{matrix} \qquad [ Equation 5 ]$$

Wherein W is an unmixing matrix of (M−1)×(M−1), and S is a signal source matrix of N×(M−1) composed of a plurality of independent components (i.e., a plurality of signal sources).

In addition, in order to extract the shape-reference voltage waveform of cardiac blood flow, the apparatus 100 of separately measuring a lung ventilation component and a cardiac blood flow component is configured to apply a Fast Fourier Transform (FFT) to each independent component Sₘ for m=1, 2, . . . , (M−1) and obtain a frequency spectrum Ψ(Sₘ) for each independent component.

Thereafter, the apparatus 100 of separately measuring a lung ventilation component and a cardiac blood flow component is configured to extract the shape-reference voltage waveform of cardiac blood flow by choosing one source signal Sₕ with the largest energy at the fundamental frequency fₕ of the heart rate among the frequency spectra acquired for each independent component.

In addition, the apparatus 100 of separately measuring a lung ventilation component and a cardiac blood flow component is configured to separate/decompose the voltage data of the lung ventilation and the voltage data of the cardiac blood flow from the acquired time series voltage data by using the source consistency theory and the extracted lung ventilation shape-reference voltage waveform and the cardiac blood flow shape-reference voltage waveform.

Meanwhile, the source consistency theory is that when there exists a single time-varying conductivity source or an independent physiological activity (e.g., lung ventilation due to breathing, cardiac blood flow due to heartbeat) inside the chest such as lung ventilation or cardiac blood flow, the shapes of all time-varying voltage channels are identical and each voltage channel has different scale factors and offsets.

Thus, the voltage data of each voltage channel influenced by all sources or independent physiological activities can be approximated as a weighted sum of shape-reference voltage waveforms of the individual sources (i.e., lung ventilation and cardiac blood flow). That is, the acquired time series voltage data includes a plurality of linear weighted sums of impedance variations occurred by physiological activities including lung ventilation and cardiac blood flow.

Accordingly, 208 voltage channels x_{l,i} for i=1, 2, . . . , 208 associated with the lung ventilation according to the source consistency theory are expressed as [Equation 6].

x_{l,i}=αᵢl+dᵢ [Equation 6]

where αᵢ and dᵢ denote a scale factor and an offset of an ith voltage channel associated with lung ventilation, respectively, and l denotes the shape-reference voltage waveform of lung ventilation extracted as a principal component.

Similarly, the 208 voltage channels x_{h,i} for i=1, 2, . . . , 208 associated with cardiac blood flow according to the source consistency theory are expressed as [Equation 7].

x_{h,i}=bᵢh+eᵢ [Equation 7]

where bᵢ and eᵢ denote a scale factor and offset of the ith voltage channel associated with cardiac blood flow, respectively, and h denotes the shape-reference voltage waveform of cardiac blood flow extracted through the independent component analysis.

Meanwhile, in the absence of excessive motion artifacts for the subject, the sum of x_{l,i} and x_{h,i} as shown in [Equation 8] is approximately equal to the voltage data of the ith voltage channel of the acquired time series voltage data.

x_{l,i}+x_{h,i}=αᵢl+bᵢh+cᵢ≈xᵢ [Equation 8]

where cᵢ denotes the sum of dᵢ and eᵢ.

In addition, using [Equation 5] to [Equation 8] and the least square method, a matrix C composed of weights including scale factors and offsets related to the calculated lung ventilation and cardiac blood flow for all voltage channels 208 associated with the acquired time series voltage data can be calculated as shown in [Equation 9].

$$C = {( {B}^{T} B )}^{- 1} {B}^{T} X \qquad [ Equation 9 ]$$

$$C = ( \begin{matrix}{a}_{1} & {a}_{2} & … & {a}_{208} \\ {b}_{1} & {b}_{2} & … & {b}_{208} \\ {c}_{1} & {c}_{2} & … & {c}_{208}\end{matrix} )$$

$${B}^{T} = ( \begin{matrix}← & {h}^{T} & \to \\ ← & {I}^{T} & \to \\ I & … & 1\end{matrix} )$$

In addition, the apparatus 100 of separately measuring a lung ventilation component and a cardiac blood flow component is configured to compute the voltage data of lung ventilation and the voltage data of cardiac blood flow for each voltage channel of the acquired time series voltage data by using the computed matrix C, thereby separating the acquired time series voltage data into the voltage data of lung ventilation and cardiac blood flow. That is, the apparatus 100 of separately measuring a lung ventilation component and a cardiac blood flow component is configured to compute weights including scale factors and offsets associated with lung ventilation and cardiac blood flow for each voltage channel constituting the acquired time series voltage data, compute the voltage data for lung ventilation and cardiac blood flow from the voltage data for each voltage channel by using the computed weights, and thereby separating the acquired time series voltage data into the voltage data for lung ventilation and cardiac blood flow.

The separated voltage data corresponding to lung ventilation and cardiac blood flow are expressed as [Equation 10].

$${X}_{l} = ( \begin{matrix}↑ & ↑ & … & ↑ \\ {x}_{l , 1} & {x}_{l , 2} & … & {x}_{l , 208} \\ ↓ & ↓ & … & ↓\end{matrix} ) \qquad [ Equation 10 ]$$

$${X}_{h} = ( \begin{matrix}↑ & ↑ & … & ↑ \\ {x}_{h , 1} & {x}_{h , 2} & … & {x}_{h , 208} \\ ↓ & ↓ & … & ↓\end{matrix} )$$

where Xₗ denotes the separated voltage data for lung ventilation, and Xₕ denotes the separated voltage data for cardiac blood flow.

Meanwhile, in the present invention, since the offset dᵢ associated with lung ventilation and the offset eᵢ associated with cardiac blood flow are irrelevant in time-difference EIT image reconstruction, the value (e.g., dᵢ=eᵢ=0.5cᵢ) is preset.

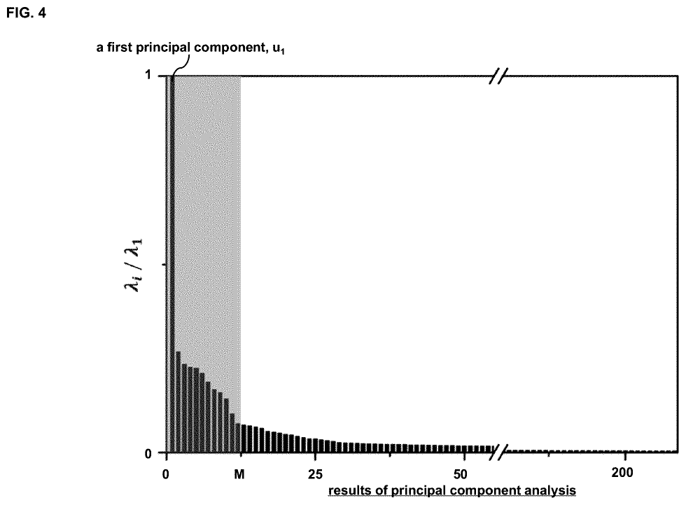

FIG. 4 is a diagram illustrating a distribution of singular values obtained by performing principal component analysis according to an embodiment of the present invention.

As shown in FIG. 4, as a result of applying the principal component analysis according to an embodiment of the present invention, the singular values for the plurality of principal components for the matrix X of the acquired time series voltage data are normalized and the normalized singular values are aligned in descending order.

In this case, the normalization is calculated as each singular value divided by a specific singular value (e.g.,) λⱼ/λₗ for j=1, 2, . . . , 208).

Thereafter, the apparatus 100 of separately measuring a lung ventilation component and a cardiac blood flow component is configured to extract the first principal component with the largest singular value, thereby extracting the shape-reference waveform associated with lung ventilation from the time series voltage data.

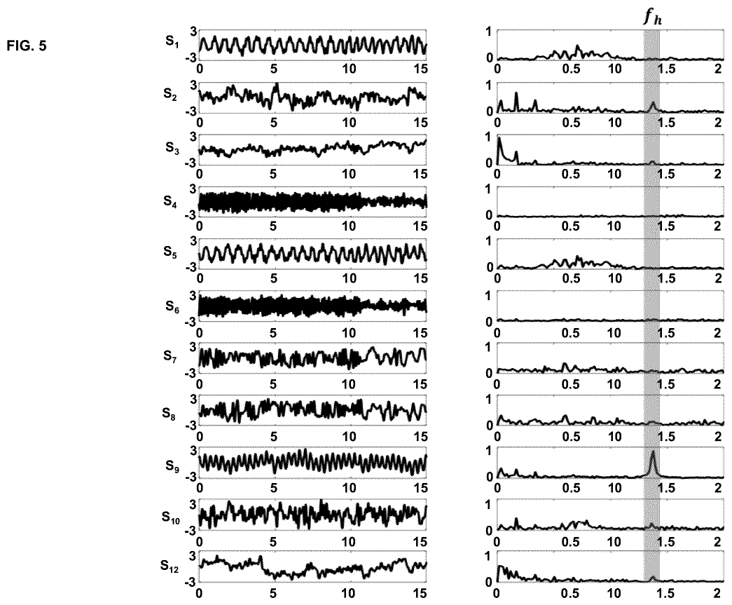

FIG. 5 is a diagram illustrating a method of extracting a cardiac blood flow shape-reference waveform by performing independent component analysis according to an embodiment of the present invention.

As shown in FIG. 5, the apparatus 100 of separately measuring a lung ventilation component and a cardiac blood flow component according to an embodiment of the present invention is configured to compute a Fast Fourier Transform (FFT) on a plurality of independent components (e.g., S₁ to S₁₁), which are the results of applying independent component analysis, and thus obtaining a frequency spectrum for each independent component.

In this case, it is preferable that the independent component analysis is applied to a preset number of principal components (e.g., 11 principal components) except for the extracted first principal component among the principal components for the plurality of singular values aligned in descending order.

The apparatus 100 of separately measuring a lung ventilation component and a cardiac blood flow component is configured to select an independent component (S₉) with the largest energy within fundamental frequency spectrum range (fₕ as shown in FIG. 5) of the heartbeat rate among the frequency spectra for each of the acquired independent components to extract the shape-reference waveform h of cardiac blood flow.

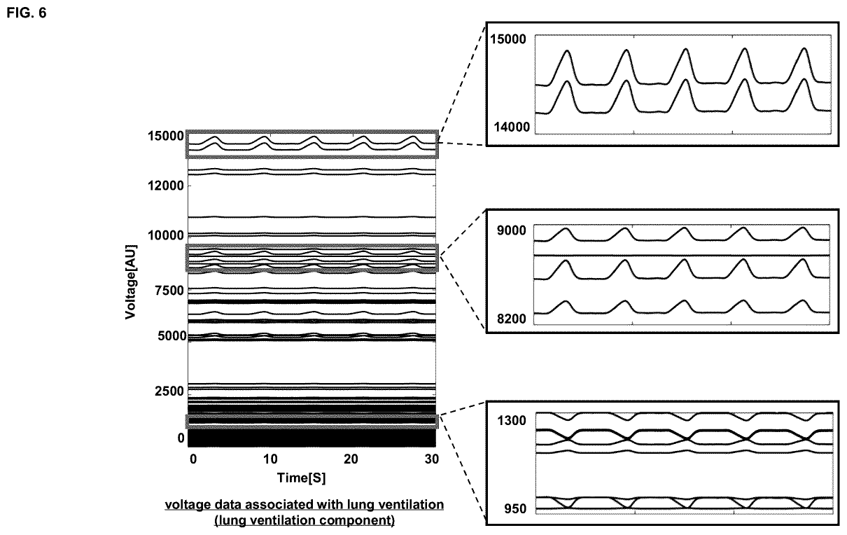

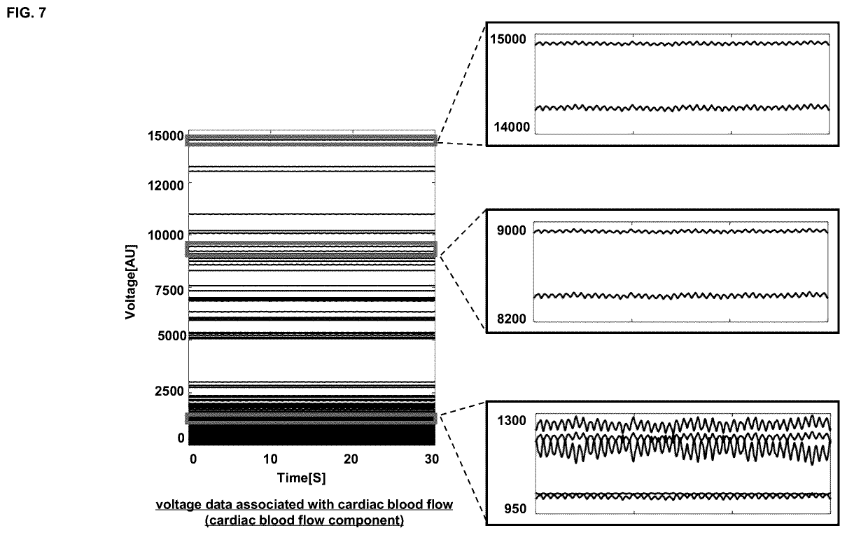

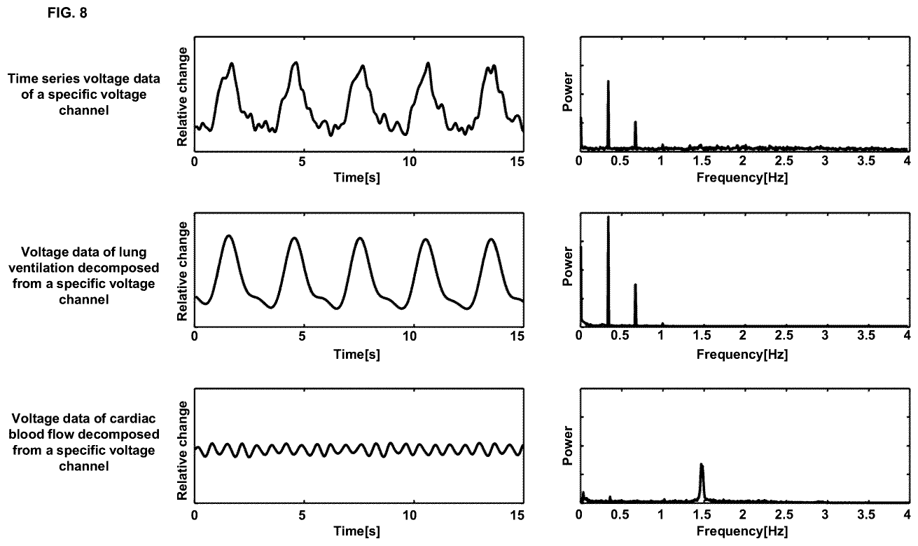

FIG. 6 is a view illustrating lung ventilation component according to an embodiment of the present invention, and FIG. 7 is a view illustrating cardiac blood flow components according to an embodiment of the present invention. FIG. 8 is a diagram illustrating voltage data, lung ventilation, and cardiac blood flow components for each voltage channel according to an embodiment of the present invention.

As shown in FIGS. 6 to 8, the voltage data associated with lung ventilation and cardiac blood flow separated from the time series voltage data acquired from a subject according to an embodiment of the present invention is separated and configured by each voltage channel of the acquired time series voltage data.

Since the separating from the acquired time series voltage data into the voltage data for lung ventilation and cardiac blood flow has been described with reference to FIG. 3 to FIG. 5, the detailed description thereof is skipped herein.

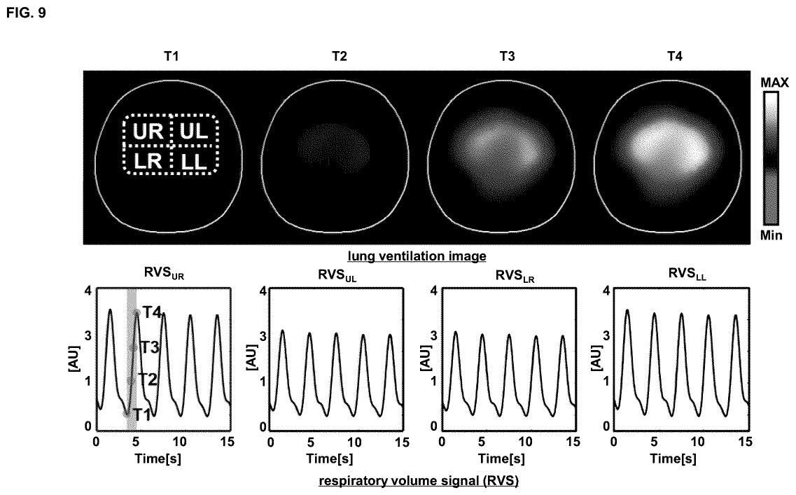

FIG. 9 is a diagram illustrating reconstructed image of lung ventilation and a respiratory volume signal according to an embodiment of the present invention.

As shown in FIG. 9, the image of lung ventilation according to an embodiment of the present invention is reconstructed by using the voltage data for lung ventilation separated from the sequentially acquired time series voltage data. Each of the images of lung ventilations shown in FIG. 9 is reconstructed by using the voltage data for lung ventilation separated from the time series voltage data acquired during the time interval (e.g., 1 second) of T₁ to T₄.

The respiratory volume signal is extracted from the reconstructed image of lung ventilation or from the separated voltage data for lung ventilation, as described above. In this case, the respiratory volume signal is extracted with respect to the entire lung region in which the region of interest is set and/or is extracted for each region of interest.

The tidal volume is measured for each breathing cycle, respectively and measured by computing valley-to-peak values of the extracted respiratory volume signal, as described above.

Meanwhile, T₁ to T₄ shown in a respiratory volume signal (RVS_{UR}) extracted from the left upper lung region (UR) shown in FIG. 9, correspond to the time period of the time series voltage data, and the valley of the respiratory volume signal indicates end expiratory having almost no air volume in the corresponding lung reason by respiration of the subject, and the peak of the respiratory volume signal indicates end inspiratory having the maximum amount of air in the corresponding lung region area by respiration of the subject.

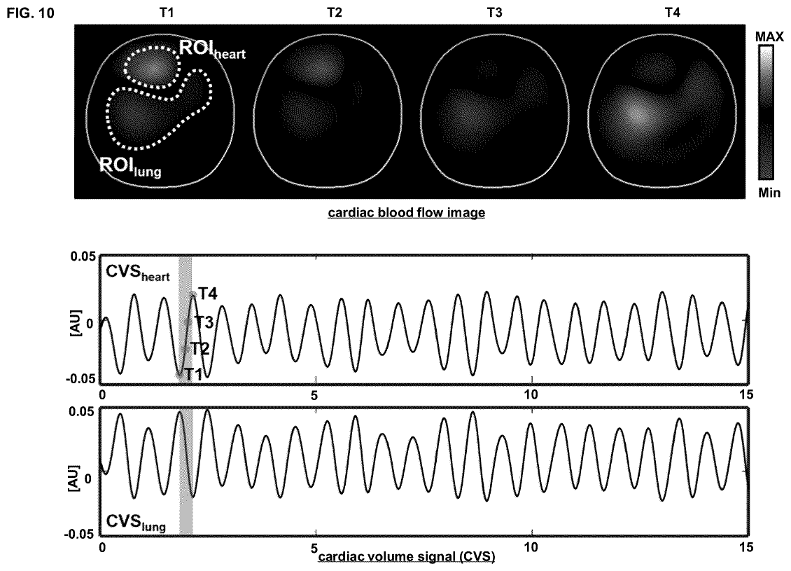

FIG. 10 is a diagram illustrating a cardiac blood flow image and a cardiac volume signal according to an embodiment of the present invention.

As shown in FIG. 10, the cardiac blood flow image according to an embodiment of the present invention is reconstructed by using the voltage data of cardiac blood flow separated from the time series voltage data continuously acquired during the time interval, T₁ to T₄.

In addition, the cardiac volume signal (CVS) is extracted from the reconstructed image of cardiac blood flow or the separated voltage data of cardiac blood flow as described above. In this case, the stroke volume is measured for each heartbeat cycle by computing a valley-to-peak values the cardiac volume signal for the extracted heart region, respectively. T₁ to T₄ shown in the cardiac volume signal extracted for the heart region shown in FIG. 10 correspond to the time interval for the acquired time series voltage data.

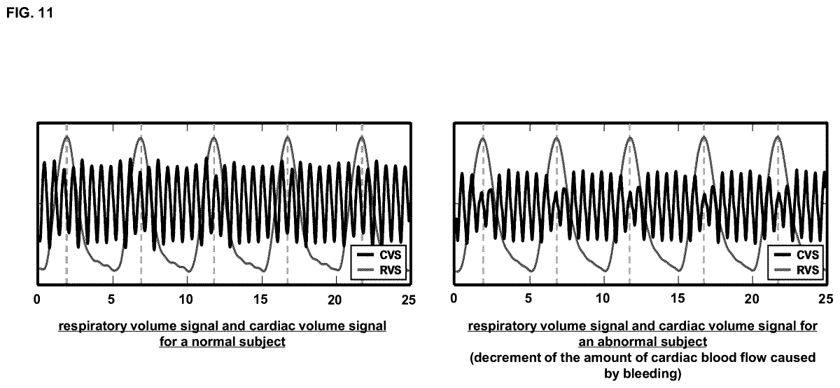

FIG. 11 is a diagram illustrating overlapping a respiratory volume signal and a cardiac volume signal of a normal subject and an abnormal subject according to an embodiment of the present invention.

As shown in FIG. 11, in comparison with the respiratory volume signal and the cardiac volume signal of the normal subject (normovolemia(left)) and those of the abnormal subject caused by arbitrary bleeding (hypovolemia(right)) according to an embodiment of the present invention, it can be seen that the amount of cardiac blood flow of the abnormal subject caused by bleeding is less than that of cardiac blood flow of the normal subject.

In addition, the apparatus 100 of separately measuring a lung ventilation component and a cardiac blood flow component is configured to overlap the extracted respiratory volume signal and cardiac volume signal over time and compute the stroke volume variation (SVV) according to [Equation 11].

$$SVV = \frac{{SV}_{max} - {SV}_{min}}{( {SV}_{max} + {SV}_{min} ) / 2} \times 100 ( \% ) \qquad [ Equation 11 ]$$

The stroke volume variation is computed for each of a plurality of preset breathing cycles (e.g., four breathing cycles). Wherein SVₘₐₓ denotes a maximum stroke volume computed during the plurality of breathing cycles, and SVₘᵢₙ denotes a minimum stroke volume computed during the plurality of breathing cycles.

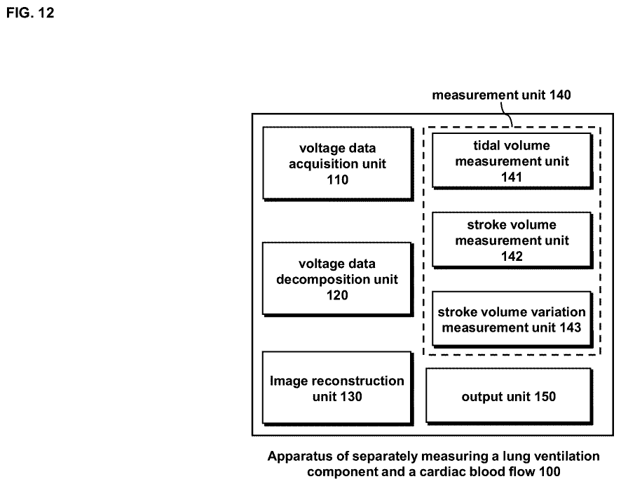

FIG. 12 is a block diagram illustrating a configuration of an apparatus of noninvasively and separately measuring lung ventilation component and a cardiac blood flow component according to an embodiment of the present invention.

As shown in FIG. 12, the apparatus 100 of separately measuring lung ventilation component and a cardiac blood flow component according to an embodiment of the present invention comprises a voltage data acquisition unit 110 configured to acquire time series voltage data from a plurality of voltage channels formed through a plurality of electrodes attached to a chest of a subject; a voltage data decomposition unit 120 configured to decompose/separate the acquired time series voltage data into the voltage data for lung ventilation and cardiac blood flow, respectively, an image reconstruction unit 130 configured to reconstruct the separated voltage data for lung ventilation and cardiac blood flow into images of lung ventilation and cardiac blood flow, respectively, a measurement unit 140 configured to measure changes/variations in lung ventilation and changes in cardiac blood flow from the separated voltage data of lung ventilation and cardiac blood flow, or the reconstructed images of lung ventilation and cardiac blood flow, and an output unit 150.

The measurement unit 140 is further configured to comprise a tidal volume measurement unit 141 configured to measure tidal volume by using the reconstructed image of lung ventilation, a stroke volume measurement unit 142 configured to measure stroke volume by using the reconstructed image of cardiac blood flow, and a stroke volume variation measurement unit 143 configured to measure stroke volume variation by using the measured tidal volume and the measured stroke volume.

The time series voltage data is consecutively acquired over time as described above.

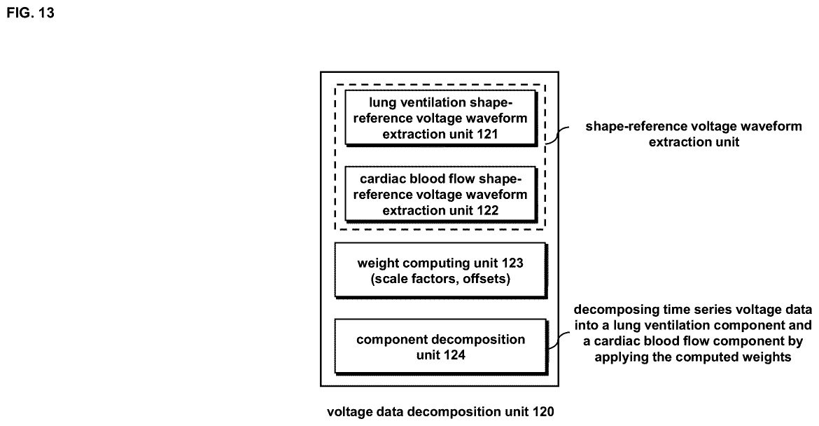

The voltage data decomposition unit 120 is configured to extracts shape-reference voltage waveforms of lung ventilation and cardiac blood flow from the acquired time series voltage data, and separate/decompose voltage data for lung ventilation and cardiac blood flow from the acquired time series voltage data by using the extracted shape-reference voltage waveforms of lung ventilation and cardiac blood flow. The configuration of the voltage data decomposition unit 120 is to be described in detail with reference to FIG. 13.

The image reconstruction unit 130 is configured to reconstruct the separated voltage data for lung ventilation into an image of lung ventilation through a predetermined image reconstruction algorithm and reconstruct the separated voltage data for cardiac blood flow into an image of a cardiac blood flow. The predetermined image reconstruction algorithm is a linearized image reconstruction algorithm using a sensitivity matrix derived from a lead field theory, as described above.

The measurement unit 140 comprises a tidal volume measurement unit 141 configured to measure tidal volume, a stroke volume measurement unit 142 configured to measure stroke volume, and a stroke volume variation measurement unit 143 configured to measure stroke volume variation.

The tidal volume measurement unit 141 is configured to extract a respiratory volume signal from the reconstructed image of lung ventilation or the separated voltage data of lung ventilation, detect a plurality of breathing cycles from the extracted respiratory volume signal, and compute tidal volume for each detected breathing cycle, thereby measuring a change in lung ventilation. The extracting of the respiratory volume signal, the detecting of the breathing cycle, and the measuring of the tidal volume have been described with reference to FIGS. 1, 3, and 9, and thus a detailed description thereof is to be skipped herein.

The stroke volume measurement unit 142 is configured to extract a cardiac volume signal from the reconstructed image of cardiac blood flow or the separated voltage data of cardiac blood flow, detect a plurality of heartbeat cycles from the extracted cardiac volume signal, and compute stroke volume for each detected heartbeat cycle, thereby measuring a change in cardiac blood flow. The extracting of the cardiac volume signal, the detecting of the heartbeat cycles, and the measuring of the stroke volume have been described with reference to FIGS. 1, 3, and 10, and thus a detailed description thereof is to be skipped herein.

The stroke volume variation measurement unit 143 is configured to overlap the extracted respiratory volume signal and the cardiac volume signal over time and measure stroke volume variation by using stroke volume having a maximum value and stroke volume having a minimum value among the stroke volumes measured by a plurality of preset breathing cycles. The measuring of the stroke volume has been described with reference to FIG. 11, and thus a detailed description thereof is to be skipped herein.

The output unit 150 is configured to recompose the reconstructed images of lung ventilation and cardiac blood flow and the measured tidal volume, the stroke volume, the stroke volume variation, or a combination thereof as graphs or images, and output the recomposed graphs and images through the display 200, and thus enabling monitoring the cardiopulmonary function of the subject in real time.

Meanwhile, the measurement unit 140 is configured to comprise outputting an alarm when the measured tidal volume, the stroke volume, and the stroke volume variation are out of preset threshold ranges of the tidal volume, the stroke volume, and the stroke volume variation. The alarm comprises visible alarm, audible alarm, or a combination thereof.

FIG. 13 is a block diagram illustrating a configuration of a voltage data decomposition unit according to an embodiment of the present invention.

As shown in FIG. 13, the voltage data decomposition unit 120 according to an embodiment of the present invention is configured to comprise a lung ventilation shape-reference voltage waveform extraction unit 121 configured to extract shape-reference voltage waveform of lung ventilation from the acquired time series voltage data, a cardiac blood flow shape-reference voltage waveform extraction unit 122 configured to extract shape-reference voltage waveform for cardiac blood flow from the acquired time series voltage data, a weight computing unit 123 configured to compute weights comprising scale factors and offsets for each voltage channel associated with the time series voltage data by using the extracted shape-reference voltage waveforms of lung ventilation and cardiac blood flow, and a component decomposition unit 124 configured to compute the voltage data for lung ventilation and cardiac blood flow for each voltage channel by applying the computed weights of lung ventilation and cardiac blood flow, respectively, and thus decompose the voltage data of lung ventilation and cardiac blood flow.

The lung ventilation shape-reference voltage waveform extraction unit 121 is configured to apply a principal component analysis for decomposing singular values for the acquired time series voltage data, choose a first principal component having the decomposed largest singular value, and extract the chosen first principal component as a shape-reference voltage waveform of lung ventilation.

In order to extract a shape-reference voltage waveform of cardiac blood flow, the cardiac blood flow shape-reference voltage waveform extraction unit 122 is configured to choose the analyzed principal components by a preset number in descending order, except for the extracted principal component among a plurality of principal components analyzed through the principal component analysis.

The cardiac blood flow shape-reference voltage waveform extraction unit 122 is configured to extract a plurality of independent components by applying independent component analysis to the chosen principal components and obtain a frequency spectrum for each of the extracted independent components by applying a fast Fourier transform to each of the plurality of the extracted independent components.

The cardiac blood flow shape-reference voltage waveform extraction unit 122 is further configured to extract the shape-reference voltage waveform of cardiac blood flow by choosing an independent component having the largest energy within the fundamental frequency range for the heartbeat rate in each obtained frequency spectrum.

The weight computing unit 123 is further configured to express the voltage data for each voltage channel associated with the acquired time series voltage data as a weighted sum of the extracted shape-reference voltage waveforms of lung ventilation and cardiac blood flow by using a source consistency theory and the extracted shape-reference voltage waveforms of lung ventilation and cardiac blood flow, and compute weights associated with lung ventilation and cardiac blood flow for each voltage channel, respectively, from the expressed weighted sum by using a least squares method. Meanwhile, the computed weights for each voltage channel are composed of a matrix, as described above.

The component decomposition unit 124 is configured to compute the voltage data for lung ventilation and cardiac blood flow for each voltage channel associated with the acquired time series voltage data by using the computed weights of lung ventilation and cardiac blood flow for each voltage channel, and thus finally decompose the acquired time series voltage data into the voltage data for lung ventilation and cardiac blood flow.

The separation/decomposition of the voltage data for lung ventilation and cardiac blood flow has been described with reference to FIG. 3, and thus a detailed description thereof is to be skipped herein.

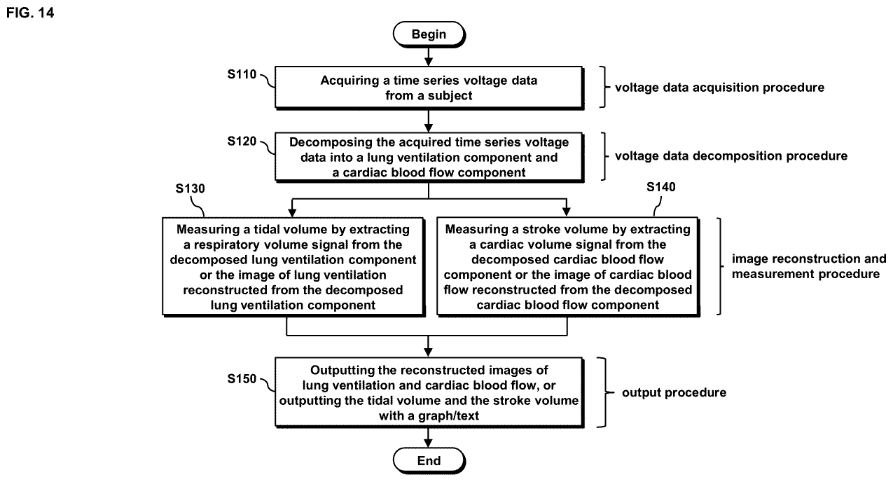

FIG. 14 is a flowchart illustrating procedures of noninvasively and separately measuring lung ventilation component and a cardiac blood flow component according to an embodiment of the present invention.

As shown in FIG. 14, in an apparatus 100 of separately measuring a lung ventilation component and a cardiac blood flow component, a procedure of noninvasively, simultaneously, and continuously measuring the lung ventilation component and the cardiac blood flow component according to an embodiment of the present invention, comprises at first acquiring time series voltage data from a subject, S110.

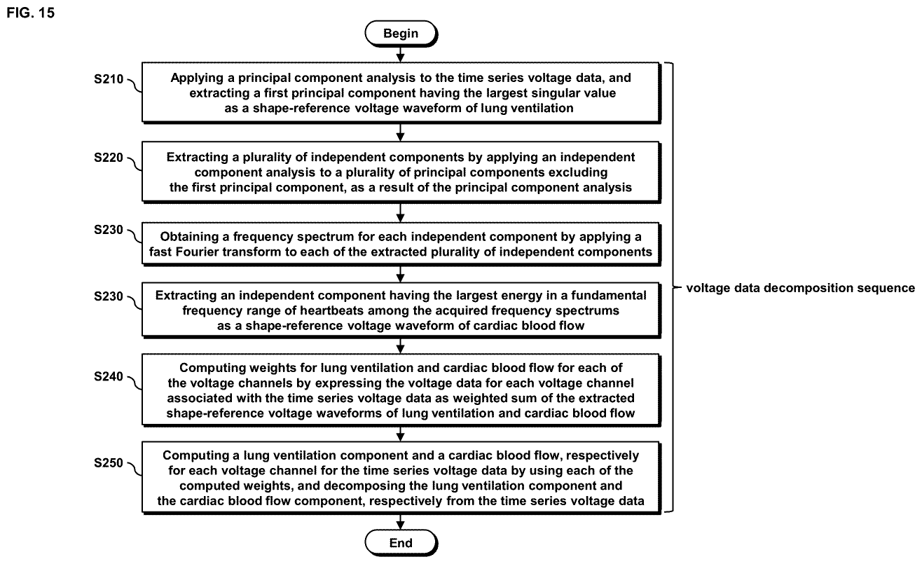

Next, in the apparatus 100 of separately measuring a lung ventilation component and a cardiac blood flow component, the procedure further comprises decomposing the voltage data of the lung ventilation component and the cardiac blood flow component from the acquired time series voltage data, S120. The decomposing/separating of the voltage data is to be described in detail with reference to FIG. 15.

Next, in the apparatus 100 of separately measuring a lung ventilation component and a cardiac blood flow component, the procedure further comprises reconstructing the separated/decomposed voltage data for lung ventilation into an image of lung ventilation, extracting a respiratory volume signal from the separated lung ventilation component or the reconstructed image of lung ventilation, and measuring tidal volume by using the extracted respiratory volume signal, S130. At the same time, the procedure further comprises reconstructing the separated/decomposed voltage data of cardiac blood flow into an image of cardiac blood flow, extracting a cardiac volume signal from the separated cardiac blood flow component or the image of cardiac blood flow, and measuring stroke volume by using the extracted cardiac volume signal, S140.

The measuring of the tidal volume is configured to measure a change in lung ventilation caused by the respiration of the subject by measuring the tidal volume according to a plurality of breathing cycles, and the measuring of the stroke volume is configured to measure a change in cardiac blood flow caused by a heartbeat of the subject by measuring the stroke volume according to a heartbeat cycle of the subject.

Meanwhile, the reconstructing and measuring of image comprises measuring stroke volume variation in the preset plurality of breathing cycles by using the extracted respiratory volume signal, the cardiac volume signal, and the measured tidal volume and stroke volume.

Next, in the apparatus 100 of separately measuring a lung ventilation component and a cardiac blood flow component, the procedure further comprises outputting the measured tidal volume, the stroke volume, and the stroke volume variation together with the reconstructed images of lung ventilation and cardiac blood flow on the display with formats of an image, a graph, or a text (numerical value), S150.

FIG. 15 is a flowchart illustrating a detailed procedure of separating voltage data according to an embodiment of the present invention.

As shown in FIG. 15, the separating/decomposing of the acquired time series voltage data from the subject into the lung ventilation component and cardiac blood flow component according to an embodiment of the present invention, first, comprises extracting a shape-reference waveform associated with lung ventilation from the acquired time series voltage data, S210.

The shape-reference voltage waveform of lung ventilation is extracted by choosing the first principal component having the largest singular value after applying a principal component analysis to the acquired time series voltage data.

Next, the separating of the voltage data comprises extracting a shape-reference voltage waveform of cardiac blood flow according to the results of applying the principal component analysis.

The extracting of the shape-reference voltage waveform of cardiac blood flow first comprises extracting a plurality of independent components by applying independent component analysis to the plurality of principal components excluding the first principal component extracted for the lung ventilation shape-reference voltage waveform as a result of applying the principal component analysis, S220. The independent component analysis is applied to the remaining principal components as many as the preset number in descending order of singular values, except for the selected first principal component as a result of applying the principal component analysis.

Next, the extracting of the cardiac blood flow shape-reference voltage waveform comprises applying fast Fourier transform to the plurality of extracted independent components and obtaining a frequency spectrum for each extracted independent component, S230, and selecting an independent component for the frequency spectrum having the largest energy within the fundamental frequency range of the heartbeat rate in the obtained frequency spectrums, and finally extracting the cardiac blood flow shape-reference voltage waveform, S230.

Next, the separating of the voltage data comprises computing weights including scale factors and offsets for lung ventilation and cardiac blood flow for each voltage channel associated with the acquired time series voltage data by using shape-reference waveforms for the extracted lung ventilation and cardiac blood flow, S240.

The weights are expressed as weighted sum of the voltage data for each of the voltage channels according to the source consistency theory for the extracted shape-reference voltage waveforms of lung ventilation and cardiac blood flow, and each weight for each voltage channel is computed from the result of the expression using a least square method, as described above.

Next, the separating of the voltage data comprises computing a lung ventilation component and a cardiac blood flow component respectively for each voltage channel associated with the acquired time series voltage data by using the computed weights, and decomposing the lung ventilation component and the cardiac blood flow component from the time series voltage data, S250.

The separating of the voltage data for the lung ventilation and the cardiac blood flow has been described with reference to FIGS. 3 and 13, and thus a detailed description thereof is to be skipped herein.

Hereinafter, an experiment result of the accuracy of tidal volume and stroke volume measured in the present invention is described by using actual tidal volume and stroke volume. The experiment was performed for 6 pigs, the actual tidal volume was measured through a mechanical artificial respirator, and the actual stroke volume was measured after correcting an actual cardiac volume signal by using a TPTD method.

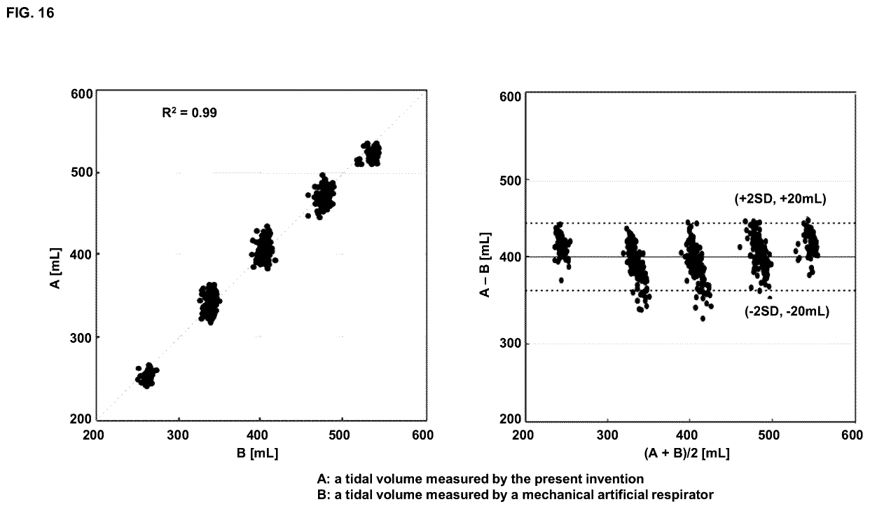

FIG. 16 is a view showing an accuracy of tidal volume according to an embodiment of the present invention.

As shown in FIG. 16, the difference between the tidal volume measured in the present invention and the tidal volume measured through a mechanical artificial respirator was −20 ml to +20 ml, and the correlation (R²) was 0.99. That is, it can be seen that the apparatus and method according to the present invention measure the tidal volume with very high accuracy.

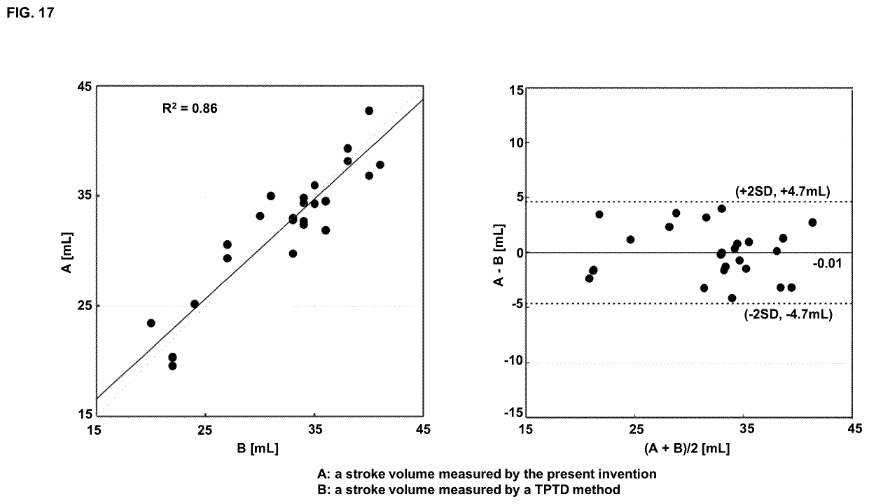

FIG. 17 is a view showing an accuracy of stroke volume according to an embodiment of the present invention.

As shown in FIG. 17, the difference between the stroke volume measured in the present invention and the stroke volume measured through the TPTD method was −4.7 ml to +4.7 ml, and the correlation (R²) was 0.86. That is, it can be seen that the apparatus and method according to the present invention measure the stroke volume with very high accuracy.

As described above, the present invention is described with reference to the embodiments and accompanying drawings, but these are merely examples, and it will be understood by a person skilled in the art that various modifications and other equivalent embodiments are possible therefrom. Therefore, the technical protection scope of the present invention should be determined by the following claims.

### Industrial Applicability

The present invention relates to an apparatus and a method of noninvasively and separately measuring a lung ventilation component and a cardiac blood flow component, and can effectively monitor the cardiopulmonary function of a subject by separating/decomposing a time series voltage data acquired from the subject into the voltage data for lung ventilation and cardiac blood flow, respectively, and then noninvasively, simultaneously and continuously measuring the tidal volume and the stroke volume.

1. An apparatus of noninvasively and separately measuring a lung ventilation component and a cardiac blood flow component, is configured to comprise:
a voltage data acquisition unit configured to acquire a time series voltage data from a subject through a plurality of voltage channels;
a voltage data decomposition unit configured to decompose the acquired time series voltage data into voltage data of lung ventilation and cardiac blood flow, which are respectively the lung ventilation component and the cardiac blood flow component; and
a measurement unit configured to measure variations in lung ventilation and in cardiac blood flow from the decomposed voltage data,
wherein the time series voltage data are composed of a linear weighted sum of impedance variations caused by a plurality of physiological activities comprising lung ventilation and cardiac blood flow.

2. The apparatus of claim 1, wherein the voltage data decomposition unit further configured to comprise:
a shape-reference voltage waveform extraction unit configured to extract shape-reference voltage waveforms associated with lung ventilation and cardiac blood flow through principal component analysis (PCA) and independent component analysis (ICA) from the time series voltage data acquired from the subject,
wherein the lung ventilation component and the cardiac blood flow component are decomposed for each voltage channel by using the extracted shape-reference voltage waveforms, and thereby the acquired time series voltage data is decomposed into the voltage data for lung ventilation and cardiac blood flow, respectively.

3. The apparatus of claim 1, wherein the apparatus further configured to comprise:
an image reconstruction unit configured to reconstruct the decomposed voltage data for lung ventilation and cardiac blood flow into images of lung ventilation and cardiac blood flow, respectively,
wherein the variations in lung ventilation and cardiac blood flow are noninvasively, simultaneously, and continuously measured from the reconstructed images of lung ventilation and cardiac blood flow.

4. The apparatus of claim 3, wherein the measurement unit further configured to extract a respiratory volume signal and a cardiac volume signal from the reconstructed images of lung ventilation and cardiac blood flow, or the decomposed voltage data of lung ventilation and cardiac blood flow, respectively, and noninvasively, simultaneously, and continuously measure tidal volume and stroke volume, thereby measuring the variations in lung ventilation and cardiac blood flow.

5. The apparatus of claim 2, wherein the shape-reference voltage waveform extraction unit further configured to comprise:
a lung ventilation shape-reference voltage waveform extraction unit configured to choose a principal component in descending order of singular values as results of applying principal component analysis on the acquired time series voltage data, and extract the chosen principal component as a shape-reference voltage waveform associated with lung ventilation; and
a cardiac blood flow shape-reference voltage waveform extraction unit configured to extract a plurality of independent components by applying independent component analysis on a plurality of principal components excluding the chosen principal component, and extract independent components associated with heartbeats among the plurality of the extracted independent components as a shape-reference voltage waveforms associated with cardiac blood flow.

6. The apparatus of claim 2, wherein the voltage data decomposition unit further configured to comprise:
a weight computing unit configured to compute weights comprising scale factors and offsets for lung ventilation and cardiac blood flow for each voltage channel associated with the acquired time series voltage data by using the extracted shape-reference voltage waveforms of lung ventilation and cardiac blood flow,
wherein the voltage data of lung ventilation and cardiac blood flow from the voltage data are computed for each voltage channel by using the weights associated with lung ventilation and cardiac blood flow computed for each voltage channel, so that the acquired time series voltage data are decomposed into the voltage data of lung ventilation and cardiac blood flow.

7. The apparatus of claim 5, wherein the cardiac blood flow shape-reference waveform extraction unit further configured to apply a Fast Fourier Transform (FFT) on each of the plurality of the extracted independent components, and obtain a frequency spectrum for each extracted independent component, and choose the independent component for the frequency spectrum having the biggest energy within the fundamental frequency range of the heartbeat rate, so that the cardiac blood flow shape-reference voltage waveform is extracted.

8. The apparatus of claim 6, wherein the weight computing unit further configured to represent the voltage data for each voltage channel of the acquired time series voltage data as a weighted sum of the extracted lung ventilation shape-reference voltage waveform and the cardiac blood flow shape-reference voltage waveform, and compute weights of lung ventilation and cardiac blood flow by applying a least square method for each voltage channel from the represented weighted sum.

9. The apparatus of claim 4, wherein the respiratory volume signal is extracted by summing all the voltage data for region of interests preset in a lung region in the decomposed voltage data of lung ventilation or pixel values for region of interests preset in a lung region in the reconstructed image of lung ventilation, respectively, and
wherein the cardiac volume signal is extracted by summing all the voltage data for region of interests preset in a heart region in the decomposed voltage data of cardiac blood flow or pixel values for region of interests preset in the heart region in the reconstructed image of cardiac blood flow, respectively.

10. The apparatus of claim 4, wherein the tidal volume is measured for each breathing cycle by computing a valley-to-peak value of each breathing cycle detected from the extracted respiratory volume signal,
wherein the stroke volume is measured for each heartbeat cycle by computing a valley-to-peak value of each heartbeat cycle detected from the extracted cardiac volume signal, and
wherein the breathing cycle and the heartbeat cycle are extracted by detecting continuous occurrence of valley-peak-valley in the extracted each respiratory volume signal and cardiac volume signal.

11. The apparatus of claim 4, wherein the measurement unit further configured to comprise mutually overlapping the extracted respiratory volume signal and cardiac volume signal over time and measuring a change in the stroke volumes according to a plurality of preset breathing cycles, by using a maximum value and a minimum value of the measured stroke volumes in the plurality of preset breathing cycles.

12. A method of noninvasively and separately measuring a lung ventilation component and a cardiac blood flow component, comprises:
acquiring a time series voltage data from a subject through a plurality of voltage channels;
decomposing the acquired time series voltage data into voltage data of lung ventilation and cardiac blood flow, which are respectively the lung ventilation component and the cardiac blood flow component; and
measuring variations in lung ventilation and in cardiac blood flow from the decomposed voltage data,
wherein the time series voltage data are composed of a linear weighted sum of impedance variations caused by a plurality of physiological activities comprising lung ventilation and cardiac blood flow.

13. The method of claim 12, wherein the decomposing of the voltage data further comprising:
extracting shape-reference voltage waveforms associated with lung ventilation and cardiac blood flow through principal component analysis (PCA) and independent component analysis (ICA) from the time series voltage data acquired from the subject,
wherein the lung ventilation component and the cardiac blood flow component are decomposed for each voltage channel by using the extracted shape-reference voltage waveforms, and thereby the acquired time series voltage data is decomposed into the voltage data for lung ventilation and cardiac blood flow, respectively.

14. The method of claim 12, wherein the method further comprising:
reconstructing the decomposed voltage data for lung ventilation and cardiac blood flow into images of lung ventilation and cardiac blood flow, respectively,
wherein the variations in lung ventilation and cardiac blood flow are noninvasively, simultaneously, and continuously measured from the reconstructed images of lung ventilation and cardiac blood flow.

15. The method of claim 14, wherein the measuring of variations further comprising:
extracting a respiratory volume signal and a cardiac volume signal from the reconstructed images of lung ventilation and cardiac blood flow, or the decomposed voltage data of lung ventilation and cardiac blood flow, respectively, and noninvasively, simultaneously, and continuously measuring tidal volume and stroke volume, thereby measuring the variations in lung ventilation and cardiac blood flow.

16. The method of claim 13, wherein the extracting of the shape-reference voltage waveforms further comprising:
choosing a principal component in descending order of singular values as results of applying principal component analysis on the acquired time series voltage data, and extracting the chosen principal component as a shape-reference voltage waveform associated with lung ventilation; and
extracting a plurality of independent components by applying independent component analysis on a plurality of principal components excluding the chosen principal component, and extracting independent components associated with heartbeats among the plurality of the extracted independent components as a shape-reference voltage waveforms associated with cardiac blood flow.

17. The method of claim 13, wherein the decomposing of the voltage data further comprising:
computing weights comprising scale factors and offsets for lung ventilation and cardiac blood flow for each voltage channel associated with the acquired time series voltage data by using the extracted shape-reference voltage waveforms of lung ventilation and cardiac blood flow,
wherein the voltage data of lung ventilation and cardiac blood flow from the voltage data are computed for each voltage channel by using the weights associated with lung ventilation and cardiac blood flow computed for each voltage channel, so that the acquired time series voltage data are decomposed into the voltage data of lung ventilation and cardiac blood flow.

18. The method of claim 16, wherein the extracting of the cardiac blood flow shape-reference waveform further comprising:
applying a Fast Fourier Transform (FFT) on each of the plurality of the extracted independent components; and
obtaining a frequency spectrum for each extracted independent component and choosing the independent component for the frequency spectrum having the biggest energy within the fundamental frequency range of the heartbeat rate, so that the cardiac blood flow shape-reference voltage waveform is extracted.

19. The method of claim 17, wherein the computing of weights further comprising:
representing the voltage data for each voltage channel of the acquired time series voltage data as a weighted sum of the extracted lung ventilation shape-reference voltage waveform and the cardiac blood flow shape-reference voltage waveform, and computing weights of lung ventilation and cardiac blood flow by applying a least square method for each voltage channel from the represented weighted sum.

20. The method of claim 15, wherein the respiratory volume signal is extracted by summing all the voltage data for region of interests preset in a lung region in the decomposed voltage data of lung ventilation or pixel values for region of interests preset in a lung region in the reconstructed image of lung ventilation, respectively, and
wherein the cardiac volume signal is extracted by summing all the voltage data for region of interests preset in a heart region in the decomposed voltage data of cardiac blood flow or pixel values for region of interests preset in the heart region in the reconstructed image of cardiac blood flow, respectively.

21. The method of claim 15, wherein the tidal volume is measured for each breathing cycle by computing a valley-to-peak value of each breathing cycle detected from the extracted respiratory volume signal,
wherein the stroke volume is measured for each heartbeat cycle by computing a valley-to-peak value of each heartbeat cycle detected from the extracted cardiac volume signal, and
wherein the breathing cycle and the heartbeat cycle are extracted by detecting continuous occurrence of valley-peak-valley in the extracted each respiratory volume signal and cardiac volume signal.

22. The method of claim 15, wherein the measuring of the variations further comprising:
mutually overlapping the extracted respiratory volume signal and cardiac volume signal over time and measuring a change in the stroke volumes according to a plurality of preset breathing cycles, by using a maximum value and a minimum value of the measured stroke volumes in the plurality of preset breathing cycles.
